SEQUENCE LISTING (2 sequences)

SEQ ID NO: 1 - DNA, 4001 bp, human
  CDS (172)..(3213)
aggcatcagcaatctatcagggaacggcggtggccggtgcggcgtgttcggtggcggctctggccgctcaggcgcctgcggctgggtgagcgcacgcgaggcggcgaggcggcagcgtgtttctaggtcgtggcgtcgggcttccggagctttggcggcagctaggggaggatggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgaccagcagaaagtcaagaagacagcggaagctggaggagtgacaggcaaaggccaggatggaattggtagcaaggcagagaagactctgggtgactttgcagcagagtatgccaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagctgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaaacaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcacttcatgaaattatatgaagaaaaaaccgggaacgcttggcactccaaaaatttcacgaagtatcccaaaaagttctaccccctggagattgactatggccaggatgaagaggcagtgaagaagctgacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggtggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacaatgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactgccatacgtctcagggagacccaataggcttaatcctgttgggagaagttgcccttggaaacatgtatgaactgaagcacgcttcacatatcagcaagttacccaagggcaagcacagtgtcaaaggtttgggcaaaactacccctgatccttcagctaacattagtctggatggtgtagacgttcctcttgggaccgggatttcatctggtgtgaatgacacctctctactatataacgagtacattgtctatgatattgctcaggtaaatctgaagtatctgctgaaactgaaattcaattttaagacctccctgtggtaattgggagaggtagccgagtcacacccggtggctctggtatgaattcacccgaagcgcttctgcaccaactcacctggccgctaagttgctgatgggtagtacctgtactaaaccacctcagaaaggattttacagaaacgtgttaaaggttttctctaacttctcaagtcccttgttttgtgttgtgtctgtggggaggggttgttttggggttgtttttgttttttcttgccaggtagataaaactgacatagagaaaaggctggagagagattctgttgcatagactagtcctatggaaaaaaccaagcttcgttagaatgtctgccttactggtttccccagggaaggaaaaatacacttccacccttttttctaagtgttcgtctttagttttgattttggaaagatgttaagcatttatttttagttaaaaataaaaactaatttcatactatttagattttcttttttatcttgcacttattgtcccctttttagttttttttgtttgcctcttgtggtgaggggtgtgggaagaccaaaggaaggaacgctaacaatttctcatacttagaaacaaaaagagctttccttctccaggaatactgaacatgggagctcttgaaatatgtagtattaaaagttgcatttgaaattcttgactttcttatgggcacttttgtcttccaaattaaaactctaccacaaatatacttacccaagggctaatagtaatactcgattaaaaatgcagatgccttctctaaaaaaaaaaaaaaaaa

SEQ ID NO: 2 - protein, 1014 aa, human
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHNAYDLEVIDIFKIEREGECQRYKPFKQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTTPDPSANISLDGVDVPLGTGISSGVNDTSLLYNEYIVYDIAQVNLKYLLKLKFNFKTSLW

(19) United States
(12) Patent Application Publication
(10) Pub. No.: US 2010/0196909 A1
(43) Pub. Date: Aug. 5, 2010

(54) PARP-BASED CYTOCHEMICAL AND HISTOCHEMICAL DETECTION METHODS AND KITS THEREFORE

(76) Inventors: Mathias Ziegler, Bergen (NO); Marc Niere, Bergen (NO); Christian Doelle, Bergen (NO)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/697,201

(22) Filed: Jan. 29, 2010

Related U.S. Application Data
(60) Provisional application No. 61/148,073, filed on Jan. 29, 2009.

(30) Foreign Application Priority Data
Jan. 29, 2009 (EP) 09001203.0

(51) Int. Cl. C12Q 1/68; C12N 15/63

(52) U.S. Cl. 435/6; 435/320.1

(57) ABSTRACT

The present invention relates to new cytochemical and histochemical detection methods based on the enzymatic activity of poly-ADP-ribose-polymerases (PARP). In particular, the present invention provides methods and systems for cytochemical or histochemical detection of a molecule of interest based on detecting poly-ADP-ribose (PAR). Further, the present invention provides methods for screening for compounds altering the amount of NAD or analogs thereof in subcellular compartments based on detecting the amount of PAR formed in said subcellular compartment as well as methods for determining the spatial distribution of a molecule of interest. In a further aspect, the present invention provides systems and kits for conducting said methods as well as a new expression vectors suitable for said systems.

### FIELD OF THE INVENTION

The present invention relates to new cytochemical and histochemical detection methods based on the enzymatic activity of poly-ADP-ribose-polymerases (PARP). In particular, the present invention provides methods and systems for cytochemical or histochemical detection of a molecule of interest based on detecting poly-ADP-ribose (PAR). Further, the present invention provides methods for screening for compounds altering the amount of NAD or analogs thereof in subcellular compartments based on detecting the amount of PAR formed in said subcellular compartment as well as methods for determining the spatial distribution of a molecule of interest. In a further aspect, the present invention provides systems and kits for conducting said methods as well as a new expression vectors suitable for said systems.

### BACKGROUND OF THE INVENTION

Biological polymers (biopolymers) are made of repetitive units, namely monomers. They are generated from relatively small molecules by enzymes such as polymerases or synthases. Polymers are composed of either different monomer entities (heterogeneous polymers) or a single type of monomeric units (homogenous polymers). Polymer generation is a common process in organisms of all natural kingdoms (bacteria, archaea, fungi, plants and animals). Biological polymers can be synthesized from different substance classes.

Polymer formation is known for various small molecules, like sugar molecules. For example, nicotinamide adenine dinucleotide (NAD) serves as substrate for poly-ADP-ribose-polymerases (PARPs), an enzyme class which cleaves the nicotinamide ring from the NAD molecules and generates polymers of the remaining ADP ribose units. For instance, the mammalian PARP enzymes are capable of generating PAR consisting of up to 200 ADP-ribose units and to attach them to specific acceptor proteins including themselves (automodification).

In general, biopolymers can consist of several hundreds to thousand individual subunits and can be branched providing the polymer with a characteristic three-dimensional structure.

Typically, the catalyzing enzymes differ in their subcellular localization. Eukaryotic organelles contain partial or complete metabolic pathways. While glycogen synthesis takes place in the cytosol and the products are stored in granular structures, starch synthesis in plants takes place within specific membrane coated organelles (plastids). Other polymerases, like DNA and RNA polymerases, exhibit their catalytic activity in specific compartments, e.g. in the nucleus, or in the mitochondria and, in plants, in the chloroplasts. The above mentioned PARPs exist in several isoforms located in different cellular compartments. In humans, the most abundant and active form, PARP1, is located to the nucleus. The distinct locations of polymerases and synthases also indicate a compartmented availability of the substrates needed for the reaction.

Antibody-based detection systems are important and prevalent methods for the specific detection of macromolecules such as proteins or biopolymers. For instance, immunoblot analyses or enzyme-linked immunosorbent assays (ELISA) of samples deriving from tissues, cells, body fluids are currently used both in molecular biology and biochemistry as well as in diagnostics to e.g. determine the abundancy of proteins, their modification state and changes in gene expression levels.

Detection is usually performed by binding of a secondary antibody that specifically detects the primary antibody bound to the antigen of interest (AOI). Most often, the secondary antibody is conjugated with an enzyme activity (e.g., alkaline phosphatase or horse radish peroxidase). This enzyme activity can be used to convert a soluble dye into an insoluble form of e.g. a different color (colorimetric detection). The more popular detection method using enzyme-conjugated secondary antibodies is the incubation with a substrate that subsequently emits light (chemiluminescent detection), which is detected either by a CCD camera that displays an image in dependence of the amount of light generated or by placing a membrane onto a film. Besides colorimetric or chemiluminescent techniques, antibodies can be conjugated with fluorescent dyes for antigen detection. Here, particularly fluorophores emitting near-infrared light have been shown to be most suitable, since membrane surfaces and biomolecules exhibit greatly reduced autofluorescence in this wavelength range.

The applicability of immunoblot analyses and ELISA is limited by the level of detection, e.g. for the identification of low abundant antigens (such as hormones) in body fluids (e.g., blood). The detection limit of proteins using fluorescent and chemiluminescent techniques can be up to the lower picogram range.

Other immunochemical techniques such as immunocytochemistry and immunohistochemistry are commonly applied to narrow down the location of the AOI in tissues or cells. Immunodetection, which is predominantly done by using enzyme-conjugated or fluorescence-labelled antibodies, allows for analyzing subcellular localization of the AOI (e.g., its association with a subcellular compartment). However, it does not enable the determination of its suborganellar distribution. The identification of silver enhanced immunogold particles by electron microscopy is a reliable antibody-based detection method that overcomes this difficulty.

The generation of antibodies using recombinant DNA technologies is impeded because of their complex molecular architecture: Antigen binding by a classical vertebrate antibody requires the variable domains of both a heavy chain and a light chain. As an important exception, antibodies from camelid and shark species were shown to lack the light chain, thus, forming single chain antibodies. The heavy chain is made up of two constant domains (CH2 and CH3) and a single variable domain (VHH), which is important for antigen binding. Due to the requirements of only one polypeptide chain to form a functional immunoglobin, these antibodies are considered a powerful tool in both therapeutic concepts and applied research, since they are suitable for a manipulation using recombinant DNA technologies and can therefore be expressed as multispecific and multifunctional fusion proteins in heterologous systems.

Knowing the precise subcellular localization of a protein is essential for the understanding of its biological function. In this regard, it is of importance to establish whether a protein is present within a specific, membrane-enclosed cell organelle. A particular challenge represent organelles with more than one surrounding membrane, such as mitochondria. Here, soluble or peripheral membrane proteins could reside in the matrix, the intermembrane space or at the outer surface (of the outer membrane). There are various methods to visualize the selective association of a molecule of interest (MOI), such as a protein of interest (POI) with subcellular compartments. These are most commonly based on overexpressed fusion proteins (part of which is the POI). One of the most widely used applications is the fusion of the POI to a fluorescent protein, such as the green fluorescent protein (GFP), which is then detected by its intrinsic fluorescence. Alternative detection methods use immunochemical techniques or specific properties of the protein or peptide fused to the POI (such as a catalytic activity). A general disadvantage of these methods consists in the lack of resolution of the suborganellar localization. That is, these methods mostly rely on (fluorescence) microscopic analyses which do not allow establishing whether the POI is located within the organelle or, for example, associated at the outer surface. At present, immunogold labelling in conjunction with electron microscopy is used to resolve this problem. Alternatively, cell or tissue disintegration and subfractionation of organelles are used to address this problem. However, these methods are labour-intensive and often inconclusive. Moreover, they are not applicable to large scale analyses.

Analyses of metabolites represent an important part of diagnostic procedures, but are also a vital instrument to understand the basic functions and advanced signalling pathways in cells. At present, comprehensive determinations of cellular, tissue or organismal metabolites are conducted to evaluate metabolite changes (metobolomics). These investigations use different methods (mass spectrometry coupled to chromatographic procedures, NMR and others), but do largely not consider subcellular metabolite pools. This limitation is, in part, caused by the necessity to obtain pure subcellular fractions which is time-consuming and error-prone.

Still, the vast majority of current knowledge regarding the subcellular distribution of small molecules (metabolites) has been established by cell fractionation and subsequent analysis of metabolites in the enriched organelle fractions. Owing to the more advanced methods of subcellular protein localization, the presence of metabolites has also been inferred from the localization of enzymes specifically converting them. Only in rare instances can metabolites be directly detected in their physiological compartments. For example, reduced pyridine nucleotides are fluorescent and they can be visualized in live cells by advanced microscopic techniques. While advanced physical methods (e.g., NMR) allow to specifically detect metabolites and their relative changes in cells or tissues, their resolution is still insufficient to resolve metabolite concentrations in subcellular structures.

Enzymes have been used as biosensors to detect metabolites, primarily in fluids, by so called enzyme based self-referencing biosensors. Despite the ongoing minimization of such devices, they are not applicable to the study of subcellular metabolite pools. In particular, glucose detection devices are of interest to monitor blood levels in diabetes patients. Based on chemical conversions, optical methods have been proposed which result in the formation of a fluorescent glucose adduct that can be detected.

In order to detect biopolymers (e.g., in biological samples), different methods can be applied. Immunochemical detection allows for specific and sensitive identification of a given macromolecule using specifically raised antibodies. The antibodies do not detect the polymer components in their monomeric state. Alternatively, the polymers can be detected by chemical modification of reactive groups of the monomeric subunits. While the use of polymerases has several technical applications, their utility as molecular detectors, for example, in cell based systems, has not been exploited. Being substrate for polymerase, in principle, the presence of the monomers within a system can be deduced from their occurrence in polymers.

Hence, there is ongoing need for new methods and tools allowing detection of a molecule of interest (MOI), like an AOI or POI, in a probe based on cytochemical or histochemical detection systems. In particular, there is ongoing need for histochemical and cytochemical analysis, in particular of immunocytochemical and immunohistochemical analysis of samples, like biological samples, allowing qualitive as well as quantative determination of the molecule of interest with increased sensitivity of the detection method.

### BRIEF DESCRIPTION OF THE PRESENT INVENTION

In a first aspect, the present invention provides a method for cytochemical or histochemical detection of a molecule of interest, comprising the steps of
a. providing a molecule having a poly-ADP-ribose-polymerase (PARP) activity linked with a molecule able to bind directly or indirectly to the molecule of interest, or nucleic acids encoding the same;
b. allowing binding of the molecule of a.) to the molecule of interest;
c. providing a substrate of the PARP enzyme and generation of poly-ADP-ribose (PAR) by enzymatic reaction of the PARP enzyme;
d. detection of PAR.

In a further aspect, the present invention provides methods for screening for compounds altering the amounts of substrates of PARP, like NAD or analogs thereof, in subcellular compartments comprising the use of a fusion construct of a catalytic moiety of PARP and a moiety directing the catalytic moiety of PARP to a predetermined subcellular compartment and determining the amount of PAR formed in a subcellular compartment of cells transfected with the fusion construct.

In addition, the present invention relates to a method for determining the spatial distribution of a molecule of interest in a cell based on the synthesis of PAR. Moreover, the present invention provides expression vectors comprising the catalytic domain of PARP downstream of a multiple cloning site allowing introduction of the molecule of interest into the multiple cloning site. Finally, the present invention relates to systems or kits for analysing the subcellular localization of metabolites as well as for cytochemical or histochemical detection of a molecule of interest comprising the use of a vector encoding the catalytic domain of PARP downstream of a molecule for targeting said catalytic domain of PARP to the predetermined subcellular compartment and/or a molecule allowing to bind to the molecule of interest.

Finally, the use of the PARP enzyme in cytochemical or histochemical detection of a molecule of interest is disclosed.

### BRIEF DESCRIPTION OF THE DRAWINGS

FIG. 1 is a scheme showing the immunodetection principle and the improved sensitivity by enzymatically generated polymers according, to the present invention. A: Immunodetection by an enzyme coupled to the secondary antibody, generating a colorful product or light, chemiluminescence. B: Immunodetection by a fluorophor coupled to the secondary antibody. C: Immunodetection of a target molecule by an immobilized antibody, followed by the binding of a fluorophor conjugated secondary antibody. D: Immunodetection of a biopolymer generated by a polymerase bound to the primary antibody, for example, via a biotin/streptavidin interaction. E: Immunodetectin of biopolymer generated by a polymerase as a part of a chimeric fusion protein, consisting of the polymerase activity and a single polypeptide chain antibody.

FIG. 2 demonstrates that the catalytic domain of PARP 1 is active in vitro and in cells:

FIG. 2A: Molecular architecture of poly-ADP-ribose-polymerase 1 and EGFP fusion constructs. MTS, mitochondrial targeting sequence. FIG. 2B: Purified PARP 1 catalytical domain (amino acids 652 to 1014) expressed in E. coli was incubated with 1 mM NAD+. Automodification of PARP1cd was detected by Western blot analysis using an antibody specific for PAR. FIG. 2C: Western blot analysis using an antibody specific for PAR after transient expression of cytosolic and mitochondrial EGFP (cytoEGFP and mitoEGFP, respectively) and EGFP-PARP1cd (cytoPARP1cd and mitoPARP1cd, respectively) fusion constructs in HeLaS3 cells reveals accumulation of PAR in cells expressing mitochondrial PARP1cd fusion proteins. FIG. 2D: Images showing HeLaS3 cells subjected to PAR immunocytochemistry 24 hours after transient transfection with mitoPARP- or cytoPARP-encoding vectors.

FIG. 3: Schematic overview of the biochemical localization approach. The PARP1cd fusion construct consists of the C-terminal 443 amino acid residues of full-length PARP 1 harbouring the catalytic domain C-terminally fused to a protein of interest (upper panel). Differently targeted PARP1cd fusion constructs give rise to distinguishable levels of PAR accumulation (lower panel). While no PAR accumulation is detectable for cytosolic fusion proteins, mitochondrial matrix localized proteins generate PAR. Levels of PAR at the lower detection limit (dotted lines) may be obtained from proteins situated in the intermembrane space of mitochondria.

FIG. 4: Subcellular compartimentalization of NAD biosynthesis The subcellular distribution of all known steps of human NAD biosynthesis as revealed herein is summarized. The majority of NAD biosynthetic enzymes localize distributed throughout the nucleus and cytosol, allowing complete pathways to occur. NAD synthetase appears to be exclusively expressed in the cytosol. Thus, amidation of NAAD to NAD is only possible in this compartment. The human NMNAT isoforms show the most variable distribution pattern, including nucleus (NMNAT1) Golgi apparatus association (NMNAT2) and mitochondrial matrix (NMNAT3). All reactions (arrows) and intermediates that may occur in the depicted organelles are indicated. Possible import of substrates for mitochondrial NAD synthesis is indicated by dashed lines.

### DETAILED DESCRIPTION OF THE PRESENT INVENTION

In a first aspect, the invention is directed to a method for cytochemical or histochemical detection of a molecule of interest comprising the steps of
a. providing a molecule having a poly-ADP-ribose-polymerase (PARP) activity linked with a molecule able to bind directly or indirectly to the molecule of interest (MOI), or nucleic acids encoding the same;
b. allowing binding of the molecule of a.) to the molecule of interest;
c. providing a substrate of the PARP enzyme and generation of poly-ADP-ribose (PAR) by enzymatic reaction of the PARP enzyme,
d. detection of PAR.

With the term “able to bind directly or indirectly to the molecule of interest” is meant herein that the PARP activity may be bound directly to the binding partner of the MOI, e.g. an antibody directed against the MOI, whereby the PARP activity is covalently bound to the antibody. Alternatively, indirect binding may be given in case where a primary antibody directed against the MOI is used and said primary antibody is detected with a secondary antibody having a PARP activity linked thereto.

Of course, steps b. and c. above may be conducted vice versa, namely, the synthesis of the polymer PAR takes places first and, thereafter, the binding of the molecule of step a. to the MOI is enabled.

In a further aspect, the present invention is related to a method for screening for compounds altering the amounts of NAD or analogs thereof in subcellular compartments comprising the steps of:
a. providing a fusion construct of a catalytic moiety of PARP and a moiety directing the catalytic moiety of PARP to a predetermined subcellular compartment;
b. providing a cell system containing the compounds of interest;
c. introducing the fusion construct into the cell system;
d. determining the amount of PAR formed in the subcellular compartment in cells of the cell systems.

Moreover, in another aspect, the present invention is directed to a method for determining the spatial distribution of a molecule of interest, comprising the steps of:
a. providing a fusion construct comprising or encoding the catalytic domain of PARP and the molecule of interest;
b. introducing the fusion construct into a cell;
c. determining the localization of the fusion construct based on the detection of generated PAR.

In addition, the present invention is directed to a method for cytochemical or histochemical detection of a molecule of interest comprising the steps of:
a. providing a molecule having a PARP activity linked with a molecule able to bind directly or indirectly to the molecule of interest;
b. allowing binding of the molecule of a.) with the molecule of interest;
c. generation and detection of PAR.

As used herein, the term “NAD or analogs thereof” refer to substrates of the poly-ADP-ribose-polymerase resulting in the generation of oligomers or polymers of the substrate analogously to the generaton of poly-ADP-ribose when using NAD as a substrate.

Further, the term PAR includes PAR analogs obtainable when using NAD analogs as substrate for PARP.

That is, the present inventors recognized that the enzyme poly-ADP-ribose-polymerase (PARP), like the human PARP, e.g. the human PARP 1, represents a powerful molecular detection tool in generating polymers of poly-ADP-ribose (PAR) which eventually are detected by known techniques, like immunochemical, chemical or physical detection methods. The synthesis of the PAR based on the PARP enzymatic activity allows to amplify a signal of the PARP molecule or any moiety linked with the PARP molecule since the polymer provides multiple sites for the binding of detector molecules like polymer recognizing antibodies or multiples sites having chemically attached fluorophores or other marker moieties.

Thus, the underlying principle allows to amplify signals and, hence, to strongly exceed the sensitivity of detection methods, in particular, histochemical and cytochemical detection methods. Thus, the present system and methods based on PARP enzymatic activity and PAR detection represents a powerful alternative for known enzyme based detection methods, like systems based on alkaline phosphotase or peroxidase.

As used herein, the term “having a poly-ADP-ribose-polymerase (PARP) activity” refers to a molecule, typically a polypeptide having at least the catalytic moiety of PARP capable of generating PAR, for example, the human PARP1 catalytic domain, aa 652 to aa 1014 of Seq. ID No. 1 or Seq. ID No. 2 derived from GeneBank Acc. No. NP 001618. The catalytic moiety of PARP allows to synthesize poly-ADP-ribose from the substrate NAD or analogs thereof, e.g. 1, N⁸-etheno NAD, 3-acetylpyridine adenine dinucleotide (Oei et al, FEBS Lett. 397, pp 17-21, 1996) or biotinylated NAD. The PARP molecule may be provided in form of a polypeptide or in form of a nucleic acid sequence encoding the polypeptide.

The term “molecule able to bind to the molecule of interest” refers to a binding agent allowing to bind to the molecule of interest, typically, via non-covalent forces. Typical examples of said molecules comprise antibodies, like polyclonal, monoclonal antibodies or single chain antibodies, or polypeptides derived from antibodies. Typically, said binding agent interacts specifically with the molecule of interest. Alternatively, the molecule able to bind to the molecule of interest may be ligand or binding partner of the moiety of interest. For example, in case that receptor molecules represent the moiety of interest, the corresponding binding partner is a ligand of said receptor.

Further, the term “moiety directing the catalytic moiety of PARP to a predetermined subcellular compartment” refers to signal sequences known in the art for targeting molecules, typically polypeptides to specific subcellular compartments. For example, said moiety is the mitochondrial targeting sequence derived from the human cytochrome C oxidase subunit 8A, GeneBank Acc. No. NP004065.

In a preferred embodiment, the PARP enzymatic activity is linked with a binding partner, like a monoclonal antibody or a single chain antibody. Preferred, the PARP is attached covalently to the binding partner. In another preferred embodiment, the binding agent of the molecule of interest is a natural ligand of the moiety of interest. In another preferred embodiment, the PARP moiety is linked with avidin or streptavidin and, in addition, the antibody, like a monoclonal antibody or a single chain antibody, is labelled with biotin (or vice versa), thus, interaction between the biotin streptavidin (avidin) molecules occurs connecting the PARP activity with the antibody. Alternatively, the fusion construct comprises a sequence encoding streptavidin or avidin.

The PARP allows the generation of the poly-ADP-ribose (PAR) polymer. The PAR polymer is typically covalently attached to the enzyme and, consequently, allows a detailed and very specific detection of the spatial distribution of the enzyme and, thus, of the molecule of interest.

As used herein, the molecule of interest includes in particular polypeptides and nucleic acid molecules. Preferably, the molecule of interest (MOI) is a polypeptide or peptide of interest (POI), in particular, an antigen of interest (AOI).

Combining the molecule of interest or, in particular, the peptide of interest, with the catalytic activity of PARP allows to determine the spatial distribution of said molecule of interest due to detection of the presence of PAR formed due to the enzymatic activity of PARP.

Additionally, for determining the spatial distribution of the molecule of interest, a fusion construct comprising the catalytic domain of PARP and the molecule of interest in form of a vector is introduced into cells. Of course, the fusion construct may already be in form of a polypeptide which may be introduced into the cells by known techniques.

After translation of the nucleic acid sequence introduced with an expression vector containing the fusion construct in case of nucleic acids, or of the fused product of the molecule of interest in the catalytic domain of PARP in form of a polypeptide, the localisation of said fused product may be determined based on PAR generation.

The fusion construct may further contain a signalling or targeting sequence allowing site specific targeting of the construct, e.g. to an organelle of the cell. These vectors may allow to manipulate NAD levels on the subcellular level. For example, a targeted expression of PARP to peroxisomes or to the ER leads to compartment specific PAR formation as well.

In contrast to current methods which determine the subcellular localisation of a POI based on the utilisation of fluorescent proteins fused to the POI and applying immunocytochemistry or subcellular fractionation, the present invention allows to determine the subcellular/suborganellar protein localisation in more detail and with less amounts of fusion product. In contrast to methods known in the art, the present method allows to determine the suborganellar localization and would not require the utilisation of cost intensive instrumentation. Based on the overexpression of a fusion protein consisting of the POI and a known polymerase protein, it is possible to amplify the signal in the presence of the substrate of the polymerase whereby the substrate may be derived from endogenous or exogenous sources, thus, allowing the detection with higher sensitivity. The polymerase activity is directed to the natural subcellular compartment of the POI. The differential presence of metabolites serving as the substrate for enzymatic generation of polymers in subcellular structures or the differential presence of enzyme activities that degrade the polymers determine whether the overexpression of the fusion construct will lead to the formation of detectable polymers. The specificity may depend on the endogenous conditions of the model system and can be modulated for example by targeted overexpression or down regulation of degrading enzymes. For example, in case of the poly-ADP-ribose glycohydrolase (PARG), which represents the enzyme responsible for degradation of PAR, an inhibitor of this enzyme may be used to inhibit any degradation of PAR.

The determination of the localisation fusion construct is achieved by the detection of the polymerase product, like PAR, generated in the living system by the polymerase reaction. Detection is achieved by known techniques, for example, by polymer specific antibodies. Whether or not detectable polymers can be formed in a specific sub-compartment is tested by overexpression of the polymerase activity endowed with a known targeting sequence directing the protein to the subcellular structure of interest.

The method and system for conducting said method is particularly applicable for identifying protein localization within membrane coated subcellular structures. For large scale approaches, a library of POI-PARP fusion constructs can be constructed and used to screen for subcellular localization of a large set of proteins.

In addition, overexpression of a PARG activity will reduce the level of PAR if the PARG construct is directed to the same subcellular structure as the POI-PARP fusion construct.

In another embodiment, the present invention relates to a method for screening for compounds altering the amounts of NAD in subcellular compartments. Said method comprises the steps of providing a fusion construct of the catalytic moiety of PARP and a second moiety linked therewith, whereby said second moiety allows directing the catalytic moiety of PARP to a predetermined subcellular compartment. When transfecting cells with said fusion construct, for example a vector as defined herein, cells are transfected and the fusion construct is expressed as a fused protein. The PARP moiety will start to polymerize ADP-ribose from its substrate NAD when sufficient NAD is present in the subcellular or suborganellar compartment, thus, producing detectable PAR molecules. In case a compound modulating NAD metabolism is introduced into or present in said system, alteration of the amounts of NAD, and consequently of formed PAR will occur, thus, allowing to identify said compounds.

The compounds to be tested may be a chemical entity or a biological entity. The compounds of interest may be already introduced in the cell system before introduction of the fusion construct or may be provided simultaneously or thereafter.

Since the presence of PAR also depends on the activity of PARG, the method is equally sensitive to detect compounds modulating PARG activity in a compartment-specific manner.

Thus, an assay based on the above method is provided allowing screening of new molecules which may be useful for treating or preventing diseases wherein NAD, PARG or PAR and the PARP enzyme are involved.

Since the polymer detection is done typically by using polymer-specific antibodies or other agents which specifically interact with the polymers, signal intensities correlate with the concentrations of the metabolite of interest, e.g. NAD. Accordingly, the method and the assay permit to reveal changes of subcellular metabolite concentrations, e.g. NAD concentrations, brought about by changes of the physiological state of the model system. Thus, it is possible to use the assay representing a simple and fast but also reliable method for identifying compounds altering the NAD pool or the activity of PARP enzyme.

PARP is known to be involved in apoptosis and in DNA repair, hence, inhibitors or compounds altering the activity of PARP may be useful for inducing apoptosis in cells, in particular, cancerous cells. Hence, said inhibitors may be useful for the treatment of cancer or other diseases involving DNA damage or abnormal proliferation of cells.

In a further aspect, the present invention relates to a system, kit or assay for analysing subcellular localization of metabolites comprising a vector comprising a nucleic acid sequence encoding the catalytic domain of PARP operably linked with a molecule allowing targeting of the catalytic domain of PARP to the subcellular compartment and means for the detection of PAR formed due to PARP activity. Optionally, the kit further comprises a vector containing a nucleic acid sequence encoding a PARG activity operably linked with the same molecule allowing targeting of the catalytic domain of PARP to the subcellular compartment or with a different molecule allowing targeting of PARG to the same or different subcellular compartment. Thus, it is possible to verify whether the presence or absence of PAR depends on the POI-PARP construct. Namely, in the presence of the PARP and the PARG construct in the same subcellular compartment will result in degradation of PAR, and, thus, will demonstrate that the PARP and the PARG construct are present in the same compartment.

Furthermore, a system, kit or assays provided for cytochemical or histochemical detection of a molecule of interest comprising i) a molecule having a poly-ADP-ribose-polymerase activity linked with a molecule able to bind directly or indirectly to the molecule of interest, or nucleic acid molecule encoding the same; ii) a substrate of the PARP enzyme optionally labelled to the marker moieties; iii) means for detecting poly-ADP-ribose formed by PARP activity. The referenced molecule having PARP activity linked with the molecule able to bind to the molecule of interest may be a molecule as described above, e.g. a single chain antibody linked with the catalytic domain of PARP or, alternatively, streptavidin or avidin as a fusion product with at least the catalytic domain of PARP.

The vector which may be used in the assay or system according to the present invention is e.g. an expression vector comprising the catalytic domain of PARP downstream of a multiple cloning site allowing introduction of the cDNA encoding the molecule of interest into the multiple cloning site. Alternatively, upstream of the catalytic domain of PARP the nucleic acid sequence encoding the binding agent (molecule able to bind to the molecule of interest) may be present, e.g. the single chain antibody, or alternatively, streptavidin or avidin. Of course, said expression vector may contain further nucleic acid sequence for expression or regulation of the vector system or the assay system.

In the system, kit or assay for cytochemical or histochemical detection of a molecule of interest, for example an antigen of interest, the molecule having a poly-ADP-ribose-polymerase activity linked with the molecule able to bind to the molecule of interest is preferably in form of a polypeptide. For example, said construct or fusion product is a fusion product of a single chain antibody or a polypeptide derived from an antibody linked with at least the catalytic domain of PARP. Alternatively, said fusion product is an antibody to which the PARP molecule is linked via covalent linkage or via e.g. the biotin-streptavidin system.

As expression vectors, commonly known expression vectors may be used, the linkage of PARP to antibodies may be achieved by known techniques. In addition, the preparation of fusion products composed of the polypeptides described herein is well known in the art.

In principle, the fusion construct, e.g. the antibody-PARP construct could be predecorated with PAR and the polymers be chemically modified with fluorophores. This would reduce the procedure of immunocytochemistry-immunohistochemistry, because only a single antibody incubation step would be required using the prelabeled antibody. Importantly, even having only a single step, the signal would exceed that of conventional immunocytochemistry involving decorated secondary antibodies due to the attachment of detector molecules to the many polymer units, thus allowing manifold signal amplification.

Typically, NAD is used as a substrate for the PARP enzyme. NAD may be labelled in advance, e.g. with a fluorophore, a radioactive label or other known labels, e.g. biotin labelled NAD is available in the art. The PARP enzyme is able to polymerize the labelled NAD thus, generating PAR having a multitude of bound biotin. Said biotin can interact with streptavidin linked to a detector, like a fluorophore, enzyme activity or radioactivity. Thus, it is possible to amplify the signal due to the binding of a plurality of streptavidin to the PAR polymer. Alternatively, the PAR polymer may be in a form allowing chemical reaction with detector molecules. For example, PAR could be chemically modified in the ribose moieties in analogy with glycoside labelling known in the art. Also polymer specific antibodies are known as described in the examples below. Using these polymer specific antibodies labelled with e.g. a fluorophore, allows a sensitive and amplified detection of the MOI.

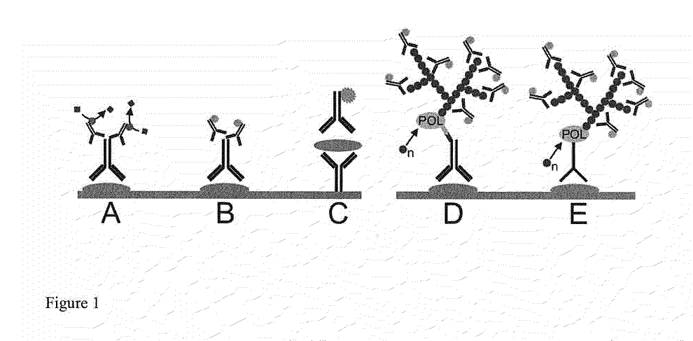

FIG. 1 provides the immunodetection principle and improved sensitivity by enzymatically generated polymers. As shown in cases D and E showing embodiments of the present invention, the signal amplification by generation of numerous epitopes for the secondary antibody in D and E shows the superiority of the system towards common immunodetection techniques. Alternatively, the polymer itself may be labelled as described above.

While embodiment E represents a fusion product of the catalytic domain of PARP and a single chain antibody, embodiment D provides an example for an biotin labelled primary antibody wherein biotin labelled PARP is attached.

In the following, a detailed description of one embodiment of the present invention is provided.

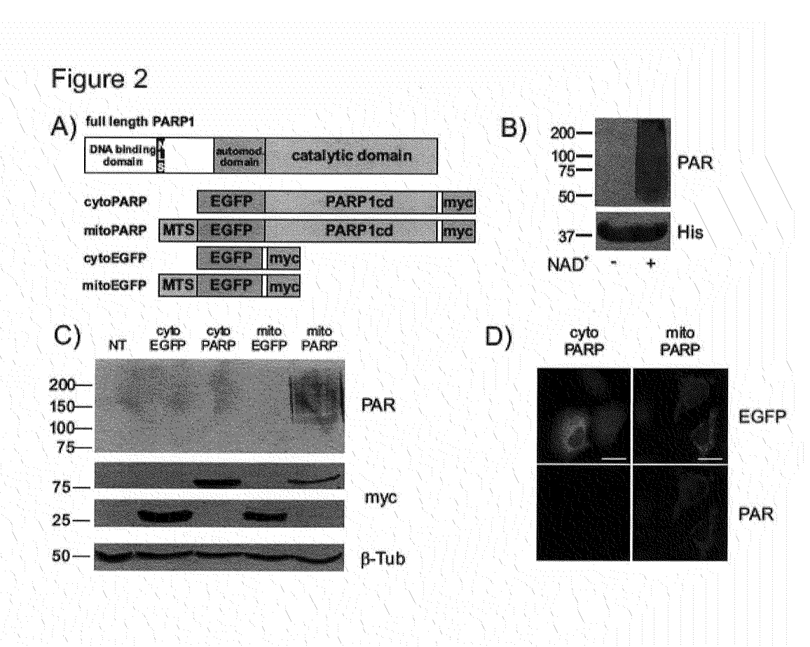

The molecular architecture of PARP1 consists of an N-terminal DNA binding domain, an automodification domain and the C-terminal part containing the catalytic domain (FIG. 2A). To verify the catalytic activity of the catalytic domain alone, a bacterially expressed, purified PARP1cd-6×His construct harbouring the amino acid residues 652-1014 has been incubated in absence and presence of the substrate NAD₊. The PARP1cd generated PAR chains and automodified itself as detected by PAR immunoblotting using 10H antibody (FIG. 2B). No polymers were detectable in absence of NAD₊.

Fusion constructs of enhanced green fluorescent protein (EGFP) and PARP1cd (residues 572 to 1014) were generated and expressed in HelaS3 cells. Expression of this protein targeted to the mitochondrial matrix by an established targeting sequence (mitoPARP) led to PAR formation readily detectable by immunoblotting (FIG. 2C). Strikingly, when the mitochondrial targeting sequence was omitted to generate a cytosolic protein (cytoPARP), no PAR formation could be observed (FIG. 2C). Likewise, the control cytosolic and mitochondrial EGFP constructs lacking the PARP1cd moiety (cytoEGFP and mitoEGFP, respectively), did not lead to any detectable PAR formation (FIG. 2C). We also closely inspected cells subjected to PAR immunocytochemistry after expressing the constructs for 24 h (FIG. 2D). Again, PAR formation was detectable in cells that expressed the mitochondrial PARP1cd construct, whereas no signal was detectable in cells that expressed the construct lacking the MTS. These observations suggested that it was the subcellular localization, and not the nature of the PARP1cd fusion protein, which determined the capability to generate detectable PAR.

To test this hypothesis, proteins with known submitochondrial localization were used. Generally, the matrix targeting sequence resides in the N-terminus of a protein. Therefore, any additional sequence at the C-terminus of the protein should not alter its mitochondrial localization. Glutamate dehydrogenase (GDH) is a well established mitochondrial matrix protein and was therefore tested. As representative of the intermembrane space (IMS), we selected apoptosis inducing factor (AIF). This protein is reported to be N-terminally anchored in the inner mitochondrial membrane and to face towards the intermembrane space and possesses an N-terminal targeting sequence.

The open reading frames encoding these proteins were cloned into an eukaryotic expression vector that added a C-terminal FLAG-epitope to the encoded proteins. The according plasmids were transiently transfected into HeLaS3 cells and the resulting proteins detected by immunocytochemistry using an antibody specific for the FLAG epitope. Co-staining with MitoTracker®Red revealed the expected association with mitochondria. Next, vectors encoding fusion constructs of GDH and AIF with the catalytic domain of PARP1 were generated. Downstream of the PARP1cd moiety the proteins contained a myc-epitope at their C-termini. Following transient transfection of the vectors, myc-immunocytochemistry and co-staining with MitoTracker®Red revealed that these constructs showed a cellular distribution just as the FLAG constructs lacking the PARP1cd.

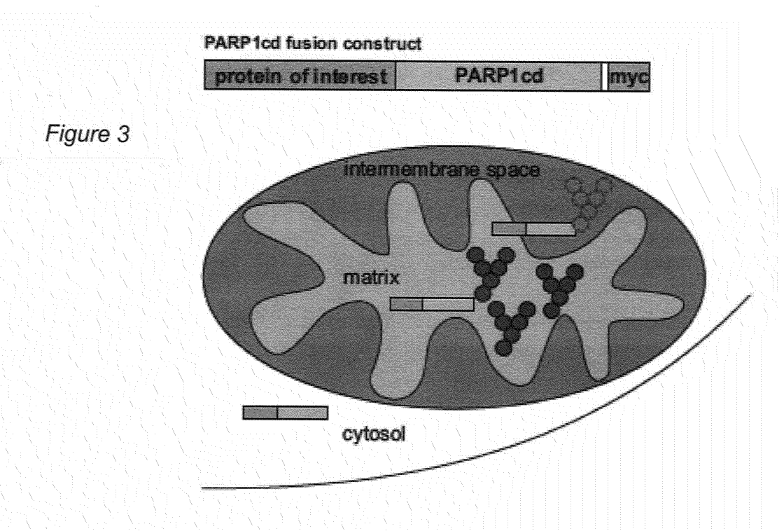

To test the capacity of these fusion proteins to generate PAR, cells have been transfected transiently with the PARP1cd fusion constructs and have been subjected to immunocytochemistry using antibodies specific for the myc epitope and PAR. The expression of the GDH-PARP1 cd construct in HeLaS3 cells resulted in detectable formation of PAR that co-localized with the myc-signal of the protein (FIG. 3). This confirmed the previous findings of mitochondrial PAR generation by a matrix localized PARP1-construct (Niere et al. supra, 2008), using an authentic matrix protein. However, the cells that expressed the IMS-targeted AIF-PARP1cd construct were PAR-negative (FIG. 3). Only a minor portion of myc-positive cells showed PAR immunoreactivity, however, only at the limit of detection.

As further evidence for the functionality of the system, a GDH-PARP1cd fusion construct was generated, which lacked the endogenous MTS encoded by the first 159 nucleotides of the open reading frame. Expression of this construct led to an accumulation of the protein in the cytosol. No detectable PAR were generated form this protein. In addition, targeted expression of AIF-PARP1cd to the mitochondrial matrix (mitoAIF-PARPcd) using the MTS of an authentic matrix protein resulted in detectable PAR formation in mitochondria.

The PAR immunoreactivity, which was exclusively observed for PARP1cd fusion proteins directed into the matrix, in fact relied on the enzymatic activity borne by PARP1cd. As exemplified for the GDH-PARP1cd fusion protein, PAR formation was no longer detectable upon treatment with the specific PARP inhibitor PJ34. Taken together, these results indicated that PARP1cd fusion may be generally used to establish whether a mitochondria-associated protein resides within the matrix.

Next, we sought to examine the subcellular distribution of key enzymes of all described NAD biosynthetic pathways in humans. Thus, expression vectors for a number of proteins as listed below have been generated. The phosphoribosyl transferases QPRT, NamPT and NAPRT as well as the two identified isoforms of nicotinamide riboside kinase all generate mononucleotide precursors of NAD. NAD synthetase converts the deamidated intermediate NAAD to NAD. As the only mitochondria-associated isoform, NMNAT3 was included in the investigation. Furthermore, the cDNA sequence of NAPRT harbors a putative alternative start codon 72 bases downstream of the original start directly downstream of an internal Kozak consensus sequence ( . . . CCACCATG . . . ), which might be used by cells to express an alternative and possibly differentially located variant of NAPRT. Therefore, the truncated sequence (NAPRTΔ1-24) was included in the analyses. Despite existing reports for several of these proteins, their subcellular distribution is still ambiguous, since, for example, only a distinction between nuclear and cytoplasmic (i.e. non-nuclear) localization could be made.

First, the subcellular distribution of these proteins carrying a C-terminal FLAG tag was investigated. NAD synthetase displayed cytoplasmic distribution, whereas QPRT, both Nrk isoforms, NamPT and NAPRT (wildtype and truncated form) were detected both in the cytoplasm and the nucleus. None of these enzymes specifically colocalized with MitoTracker®Red. In contrast, NMNAT3 revealed a cellular distribution that followed the pattern of MitoTracker®Red, which confirms the previously described association with mitochondria.

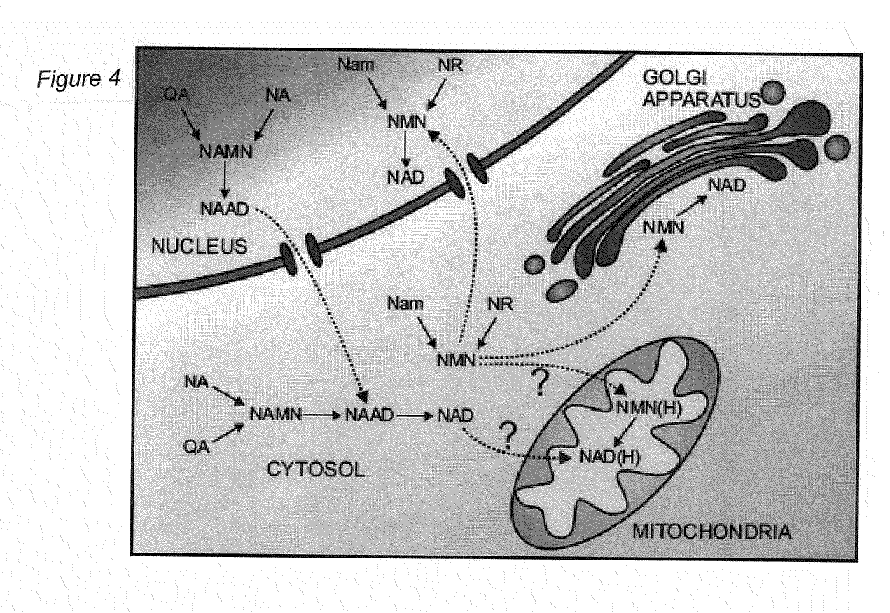

Previously, NamPT was claimed to partially localize within mitochondria, a finding that easily might rest unnoticed in a classic immunocytochemical examination. Both NamPT and NAPRT are able to provide substrates for the mitochondrial NMNAT3-catalyzed reaction from permeable precursors and could, thus, establish a viable dinucleotide synthesis pathway within this organelle. Therefore, we generated vectors encoding NMNAT3, NamPT, and NAPRT as PARP1cd fusion proteins and transiently transfected them in HeLaS3 cells. Cells expressing NMNAT3-PARP1cd fusion proteins accumulated PAR within mitochondria. This established that NMNAT3 is indeed located within the mitochondrial matrix (FIG. 4). On the other hand, NamPT and NAPRT fusion proteins maintained their cytoplasmic distribution and did not give rise to any detectable PAR formation. Consequently, none of the two proteins is located within mitochondria (FIG. 4). The PARP1cd constructs seemed to be excluded from the nucleus. These observations conclusively demonstrate that NMNAT3 is indeed located within the matrix of mitochondria.

The possibility that structural aspects of the NamPT and NAPRT fusion proteins might prevent the PARP1cd portion from being active were considered. Therefore, said molecules have been endowed with the same artificial MTS that was previously used to target PARP1cd to the matrix (Niere et al 2008, supra). Transient transfection of cells with the mitoNamPT-PARP1cd and mitoNAPRT-PARP1cd construct resulted in an exclusive mitochondrial localization pattern of the proteins. Moreover, these constructs now generated detectable PAR as a result of their matrix localization. Thus, the functionality of the constructs with regard to the PARP activity was confirmed. In addition, this experiment further substantiated the suitability of the novel import assay.

Taken together, of the known enzymes involved in NAD biosynthetic pathways, only NMNAT3 could be conclusively localized to the mitochondrial matrix.

Further, to demonstrate that other cellular compartments can be targeted, targeted expression of the catalytic domain of PARP has been performed. Namely, targeted expression of PARP1cd to the lumen of the endoplasmatic reticulum (ER), the Golgi apparatus or peroxisomes results in detectable PAR formation within these organelles. For ER targeting, the first 100 amino acids of the binding immunoglobulin protein (BiP), N-terminally fused to PARP1cd has been used. Overexpressed fusion protein was determined in the ER. With respect to the Golgi apparatus, a Golgi targeted EYEP-PARP1cd-fusion protein was used. Targeted expression of fusion protein in the peroxisomes was achieved by using an EGFP-PARP1cd fusion protein, containing the C-terminal SKL tripeptide for peroxisomal targeting. In all cases, PAR, the product of PARP was determined in the respective compartments.

Moreover, fusion constructs of antibody-PARP1cd demonstrated enhanced sensitivity of immunochemistry. Either fusion constructs of PARP1cd with the Fc part of a primary antibody as well as a fusion protein of PARP1cd with the antigen-binding domain of a single chain antibody against GFP demonstrated immunological activity, namely binding of the antigen, as well as enzyme activity, i.e. synthesis of PAR as demonstrated by fluorescence microscopy and western blot analysis, respectively.

Herein, a novel assay has been developed to study the fine localization of mitochondrial proteins and identify those that reside in the mitochondrial matrix. The accurate identification of the subcellular localization of a protein is crucial in order to understand its physiological function. Indeed the analyses of the subcellular distribution of enzymes involved in NAD biosynthesis have established that among all known proteins and isoforms only NMNAT3 is located within the mitochondrial matrix. Given the large size and important role of the mitochondrial NAD pool as well as the existence of several independent routes of NAD biosynthesis, this is a rather unexpected conclusion. All proteins analyzed in this study were localized to the cytoplasm, while some displayed an additional nuclear localization. Thus according to the presence of enzymes in these compartments, the presence and conversions of intermediates would be expected. Although never truly demonstrated, it is assumed that all the metabolites including NAD itself can freely permeate between the nucleus and the cytosol (but not the mitochondria).

The novel assay is based on a biochemical approach and relies on the observation that upon expression of the catalytic activity of PARP1, the accumulation of its product, PAR, is only detectable when the protein is present in the mitochondrial matrix. This system differs from other approaches by its simplicity, as it merely requires standard procedures of molecular and cellular biology and conventional fluorescence microscopy. The visualization of an enzymatic activity strengthens the system by reducing the possibility of false positive results, which may occur in other techniques, such as subcellular fractionation and protease treatment import assays on isolated mitochondria. Moreover, the availability of PARP1 inhibitors allows the system to be easily controlled. The application of this assay on both matrix (GDH) and intermembrane space (AIF) mitochondrial proteins clearly distinguished between differential suborganellar distributions of the analyte proteins. This stands in contrast to classic immunodetection of a protein of interest and co-staining with mitochondrial markers, which allow only for conclusions concerning an association with mitochondrial structures. The restrictions of such co-staining experiments were aimed to overcome with the present approach. The reason for the distinct PAR signal from matrix and IMS remains concealed, but restricted availability of the non-bound substrate NAD₊ outside of the matrix may be a contributing factor. With regard to cytosolically localized fusion proteins, a fast PAR turnover by cytosolic PARG could result in undetectable signals. In principle, this system is suitable to serve as a universal tool for establishing matrix localization of any given candidate proteins. Even membrane proteins embedded in the inner mitochondrial membrane may be examined with regard to their topology and orientation, provided the C-terminus protrudes on either side of the membrane.

This novel localization assay has been applied with the intention to elucidate subcellular and suborganellar distribution of those enzymatic activities that are important for, and may be directly involved in the maintenance of the mitochondrial NAD pool. An in depth analysis is required in order to determine which possible precursors might have to enter mitochondria to be finally converted to NAD in the matrix. The establishment of this separate NAD pool is of special interest due to the major redox energy metabolism and NAD degrading reactions in this compartment, involved in processes such as insulin secretion and intraorganellar protein acetylation.

### Material and Methods

### Chemicals, Reagents and Media

Unless otherwise specified, all chemicals and reagents were of analytical grade and purchased from Sigma-Aldrich and Merck. Reagents other than fetal bovine serum (Biochrom) were from Cambrex Cooperation, Nunc or Invitrogen/Gibco. PJ34 was obtained from Calbiochem. Anti-FLAG and anti-β-Tubulin antibodies were from Sigma-Aldrich, the rabbit anti-PAR antibody from Alexis Biochemicals and the chicken anti-myc antibody from Invitrogen. Mouse anti-myc antibody (9E10) and mouse anti-PAR antibody (10H) were from hybridoma cell culture supernatants. Fluorescent-conjugated secondary antibodies were of highly cross-adsorbed quality from Invitrogen/Molecular Probes. ECL reagents and HRP conjugated goat anti-mouse/goat anti-rabbit antibodies were from Pierce or GE healthcare/Amersham Biosciences. DNA modifying and restriction enzymes were purchased from Fermentas, New England Biolabs or TaKaRa, oligonucleotide synthesis was done by Sigma-Aldrich.

### Cloning and Generation of Eukaryotic Expression Vectors

The DNA sequence encoding the C-terminal catalytic domain of PARP1 (amino acids (aa) 572-1014) along with a C-terminal myc-epitope was amplified from a pre-existing vector (Niere et al., supra 2008) and ligated intopcDNA3.1(+) (Invitrogen) via EcoRI/XbaI sites. Subsequently, the open reading frames (ORFs) of the proteins of interest were amplified and introduced into the generated pcDNA3.1(+)-PARP1cd vector via KpnI/EcoRI restriction sites. In order to verify the localization of the proteins of interest by indirect immunocytochemistry, their corresponding cDNA sequences were inserted into pFLAG-CMV 5a (Sigma). In addition, the ORFs of some proteins were ligated into pCMV mito myc (Invitrogen) using the SalI restriction site. All cloned cDNAs were amplified using Pfu DNA polymerase. Recognition sites for restriction enzymes and the Kozak sequence were included in the primer sequences. For allowing targeted expression in the lumen of the endoplasmatic reticulum, the Golgi apparatus, and the peroxisomes, respectively, known targeting sequences have been used. That is, for ER targeting, the first 100 amino acids of the binding immunoglobulin protein (BiP), N-terminally fused to PARP1cd has been used. Overexpressed fusion protein was determined in the ER. With respect to the Golgi apparatus, a Golgi targeted EYEP-PARP1cd-fusion protein was used. Targeted expression of fusion protein in the peroxisomes was achieved by using an EGFP-PARP1cd fusion protein, containing the C-terminal SKL tripeptide for peroxisomal targeting. The fusion constructs of PARP1cd with either the Fc part of a primary antibody or with the antige-binding domain of a single chain antibody against GFP were expressed in bacteria and purified by their 6×HIS-tag using a Ni-NTA column.

### Cell Culture

HeLaS3 cells were cultivated in Ham's F12 medium supplemented with 10% (v/v) FCS and penicillin/streptomycin. 293 cells were cultivated in Dulbecco's modified Eagle's medium and supplemented with 10% (v/v) FCS, 2 mM glutamine and penicillin/streptomycin. Transient transfection of eukaryotic cells was performed for 24-48h using Effectene reagent (Qiagen) according to the recommendations of the manufacturer.

### Immunocytochemistry

Cells grown on cover slips were fixed with ice cold 4% (v/v) formaldehyde in PBS for 45 min and subsequently permeabilized for 15 min using 0.5% (v/v) Triton X-100 in PBS. In some experiments, cells were treated with 0.2 μM MitoTracker®Red CMXRos (Invitrogen) in full medium for 30 min prior to fixation. After blocking with complete medium for 1 h at room temperature (RT), cells were incubated with primary antibodies diluted in complete medium for ≧1 h at RT or overnight at 4° C. Cells were washed once with 0.1% (v/v) Triton X-100 in PBS and three times with PBS followed by a 1 h incubation at RT with secondary antibody diluted 1:1000 in complete medium. After staining the nuclei with DAPI, the cells were washed three times with PBS and the cover slips were mounted onto slides. Samples were analyzed on a Leica DMI 6000B microscope (Leica Microsystems).

### Protein Determination, SDS-Page and Western Blot Analysis

Protein concentration determination of cell lysates prepared with 20 mM Tris/HCl pH 7.4, 150 mM NaCl, 2% SDS and 1 mM EDTA as lysis buffer was done using a bicinchoninic acid kit (Pierce). Sodium dodecyl sulfate poly acrylamide gel electrophoresis (SDS-PAGE) and immunoblotting were carried out according to standard procedures; enhanced chemiluminescence was used for detection. For analysing PARP1cd activity of the fusion proteins, purified proteins were incubated for 30 min at 30° C. with or without 2 mM NAD⁺. Samples were separated by SDS-PAGE, and polymers were detected using PAR immunoblotting with a PAR specific antibody (9E10). The activity of PARP was maintained in the fusion proteins.

The same is true for the binding activity of the antigen-binding domains present in the fusion protein as demonstrated by fluorescence microscopy using appropiate antibodies and labels.

1. A method for cytochemical or histochemical detection of a molecule of interest comprising the steps of:
a. providing a molecule having a poly-ADP-ribose-polymerase (PARP) activity linked with a molecule able to bind to the molecule of interest, or nucleic acids encoding the same;
b. allowing binding of the molecule of a.) to the molecule of interest;
c. providing a substrate of the PARP enzyme and generation of poly-ADP-ribose (PAR) by enzymatic reaction of the PARP enzyme;
d. detection of PAR.

2. A method for screening for compounds altering the amounts of NAD in subcellular compartments comprising the steps of:
a. providing a fusion construct of a catalytic moiety of PARP and a moiety directing the catalytic moiety of PARP to a predetermined subcellular compartment;
b. providing a cell system and the compounds of interest;
c. introducing the fusion construct into the cell system;
d. determining the amount of PAR formed in the subcellular compartment in cells of the cell systems.

3. A method for determining the spatial distribution of a molecule of interest, comprising the steps of:
a. providing a fusion construct comprising the catalytic domain of PARP and the molecule of interest;
b. introducing the fusion construct into a cell;
c. determining the localization of the fusion construct based on the presence of PAR.

4. A method for cytochemical or histochemical detection of a molecule of interest comprising the steps of:
a. providing a molecule having a PARP activity linked with a molecule able to bind to the molecule of interest;
b. allowing binding of the molecule of a.) with the molecule of interest;
c. detection of PAR.

5. The method according to claim 1, wherein the molecule is able to bind to the molecule of interest is a polypeptide derived from an antibody.

6. The method according to claim 5 wherein the polypeptide is a single chain antibody.

7. The method according to claim 1 wherein the substrate of the PARP is NAD or a functional analog thereof, optionally labelled with a label or marker moiety.

8. The method according to claim 1 wherein the detection of PAR is effected by immunocytochemical or immunohysto-chemical methods, in particular, antibody based methods whereby said antibodies are labelled with a marker moiety.

9. The method according to claim 2 wherein the fusion construct is in form of an expression vector.

10. The method according to claim 1 wherein the substrate for the PARP enzyme is provided exogenously.

11. The method according to claim 1 wherein an inhibitor of the poly-ADP-ribose glycohydrolase is present.

12. The method according to claim 2 for identifying compounds for inducing apoptosis in cells.

13. The method according to claim 2 for identifying compounds for the treatment of cancer.

14. An expression vector comprising the catalytic domain of PARP downstream of a multiple cloning site and i) a DNA encoding the molecule of interest upstream of the multiple cloning site, or ii) a molecule able to bind to the molecule of interest upstream of the multiple cloning site.

15. System or kit for analysing subcellular localization of metabolites comprising
a vector comprising a nucleic acid sequence encoding the catalytic domain of PARP operably linked with a molecule allowing targeting of the catalytic domain of PARP to the subcellular compartment,
optionally, a vector comprising a nucleic acid sequence encoding PARG operably linked with the same molecule allowing targeting of the catalytic domain of PARP to the subcellular compartment or with a different molecule allowing targeting of PARG to the same or different subcellular compartment and
means for detection of PAR.

16. System or kit for cytochemical or histochemical detection of a molecule of interest comprising i) a molecule having a poly-ADP-ribose-polymerase activity linked with a molecule able to bind to the molecule of interest or nucleic acid molecule encoding the same; ii) a substrate of the PARP enzyme optionally labelled with a marker moiety; iii) means for detecting poly-ADP-ribose.

17. The use of the PARP enzyme(s) in cytochemical or histochemical detection of a molecule of interest.
